(19) United States
(12) Patent Application Publication
(10) Pub. No.: US 2008/0221559 A1
(43) Pub. Date: Sep. 11, 2008

(54) Calibration and Quality Assurance System For Use With Ophthalmic Surgical Devices and Associated Methods

(76) Inventors: Phuoc Khanh Nguyen, Winter Springs, FL (US); Alex Sacharoff, Oviedo, FL (US); James Strobel, Titusville, FL (US); Sebastian Spoerlein, Egling (DE)

Correspondence Address: ALCON, IP LEGAL, TB4-8, 6201 SOUTH FREEWAY, FORT WORTH, TX, 76134, US

(21) Appl. No.: 11/682,628

(22) Filed: Mar. 6, 2007

(51) Int. Cl. A61F 9/008

(52) U.S. Cl. 606/11

(57) ABSTRACT

A method for determining a peak fluence of an excimer laser includes scanning an excimer laser beam in a predetermined pattern on a film. The film can include a polymer layer atop a substrate including an upper layer having a first characteristic and a lower layer having a second characteristic detectably distinct from the first characteristic. The predetermined pattern includes a plurality of discrete points receiving incrementally greater numbers of pulses, at least one point receiving a sufficient number of pulses to penetrate through the polymer layer and the upper layer. A breakthrough point is detected at which a smallest number of pulses was sufficient to penetrate through the polymer layer and the upper layer, thereby exposing the lower layer to a detection of the second characteristic. A peak fluence of the excimer laser can then be determined from the number of pulses received at the detected breakthrough point.

### FIELD OF THE INVENTION

The present invention is directed to calibration systems for ophthalmic laser surgery system.

### BACKGROUND OF THE INVENTION

Precise calibration of laser fluence levels is critical for successful outcomes in ophthalmic laser systems. At present fluence testing is performed with the use of a polymer film such as Mylar®, along with an attenuator to reduce the fluence by approximately 33% from the clinically used level. The attenuator must be placed into the beam path near the laser cavity, which requires removal of the laser system covers and bed, making the test difficult.

Optics uniformity testing at present is performed by ablating a PTK pattern onto a piece of paper, such as Zap-it™ paper. The pattern formed is inspected for non-uniformities in lightness and darkness by an expert who makes a subjective judgment as to optics wear.

### SUMMARY OF THE INVENTION

The present invention is directed to a system and method for determining a peak fluence of an excimer laser. The method comprises the step of scanning an excimer laser beam comprising a series of pulses in a predetermined pattern on a film. The film can comprise a polymer layer atop a substrate comprising an upper layer having a first characteristic and a lower layer beneath the upper layer having a second characteristic detectably distinct from the first characteristic. The predetermined pattern comprises a plurality of discrete points receiving incrementally greater numbers of pulses, at least one point receiving a sufficient number of pulses to penetrate through the polymer layer and the upper layer.

A breakthrough point is detected at which a smallest number of pulses was sufficient to penetrate through the polymer layer and the upper layer, thereby exposing the lower layer to a detection of the second characteristic. A peak fluence of the excimer laser can then be determined from the number of pulses received at the detected breakthrough point.

Another aspect of the invention is directed to a method of characterizing an optical system for uniformity. This method can comprise the step of directing an excimer laser beam comprising a series of pulses through an optical train adapted to scan a substrate in a predetermined pattern. The excimer laser beam pulses are scanned in the predetermined pattern on a film as described above. The predetermined pattern can comprise a plurality of discrete points receiving a same number of pulses. The pulse number comprises a previously determined number known to be sufficient to penetrate through the polymer layer and the upper layer, thereby exposing the lower layer to a detection of the second characteristic.

The film can be examined to determine any points not having experienced a penetration through the polymer layer and the upper layer. From this examination, a correlation can be made of the non-penetrated points with an area on the optical train to identify a region of non-uniformity thereon.

### BRIEF DESCRIPTION OF THE DRAWING

FIG. 1 is a system schematic of an exemplary embodiment of the system of the present invention.

FIG. 2 is a cross-sectional view of an exemplary film of the present invention, also illustrating the determination of a beam profile.

FIG. 3 is a schematic diagram of a grid indicating an exemplary number of pulses delivered to each point thereon.

FIGS. 4-9 are photographs of exemplary film samples; FIGS. 4-6 are samples scanned at 100 Hz; FIGS. 7-9, at 400 Hz.

### DETAILED DESCRIPTION OF THE PREFERRED EMBODIMENTS

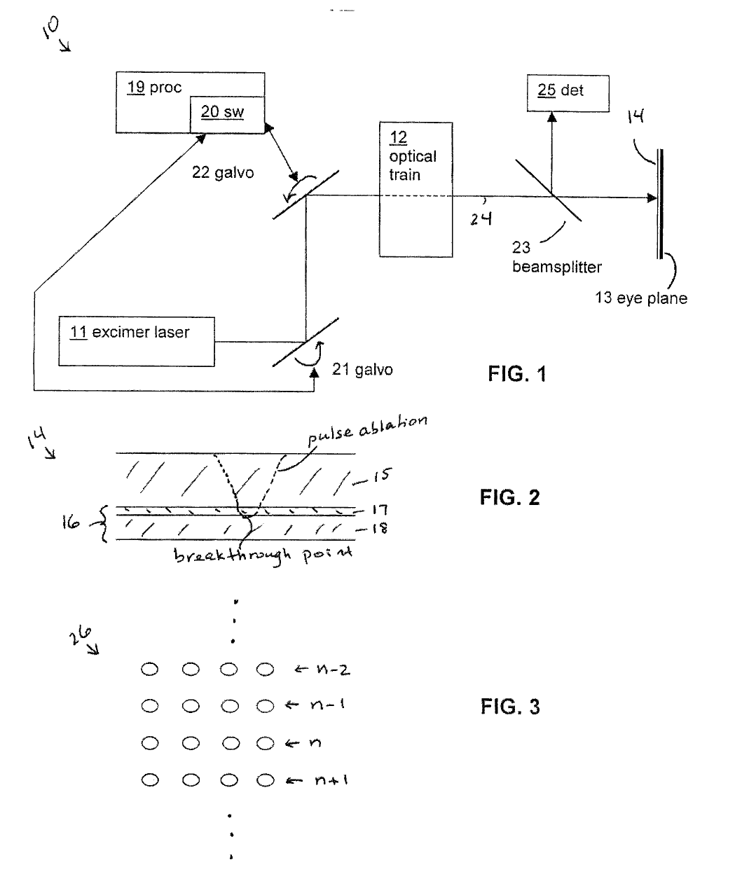

A description of preferred embodiments of the invention will now be presented with reference to FIGS. 1-9. An exemplary system 10 diagram for determining a peak fluence of an excimer laser 11 is given in FIG. 1, wherein the excimer laser 11 is positioned to deliver a series of pulses through an optical train 12 to an eye plane 13, at which is positioned a test film 14, a cross-sectional view of which is given in FIG. 2.

The film 14 can comprise a polymer layer 15 atop a substrate 16. The polymer layer 15 can comprise, for example, a Mylar® film of depth 3.5-15 μm, although this is not intended to be limiting. The substrate 16 can comprise an upper layer 17 having a first characteristic and a lower layer 18 beneath the upper layer 17 having a second characteristic detectably distinct from the first characteristic. The polymer layer 15 thickness should preferably be selected to be adequate for achieving a discrimination of at least 2-3% on the relative transmission, and most preferably approximately 1%.

In a particular embodiment, the lower layer 18 can comprise a stiff backing layer including, for example, plastic, which has a dark color. The upper layer 17 can comprise a metal film, for example, an aluminum film electrodeposited onto the polymer layer 15. In this embodiment, the first characteristic comprises a reflective surface of the aluminum film 17, and the second characteristic comprises the plastic material 18 having a color contrastive with the aluminum film surface 17, for example, a dark color such as black.

The method comprises the step of scanning in a predetermined pattern on the film 14. The scanning can be implemented by a processor 19 having software 20 resident thereon for controlling a pair of galvos 21,22. A beam splitter 23 upstream of the film 14 sends a portion of the beam 24 to a detector 25, for example, a charge-coupled-device detector, although this is not intended as a limitation.

The predetermined pattern can comprise, for example, a plurality of discrete points on a grid receiving incrementally greater numbers of pulses. For example, a grid 26 of points can be established wherein, for each row 27, one more pulse is delivered (FIG. 3) than the number delivered to the preceding row. The number n is established so that at least one point receives a sufficient number of pulses to penetrate through the polymer layer 15 and the upper layer 17 (see, for example, FIG. 2). A typical value for n could be 40, although this is not intended as limiting.

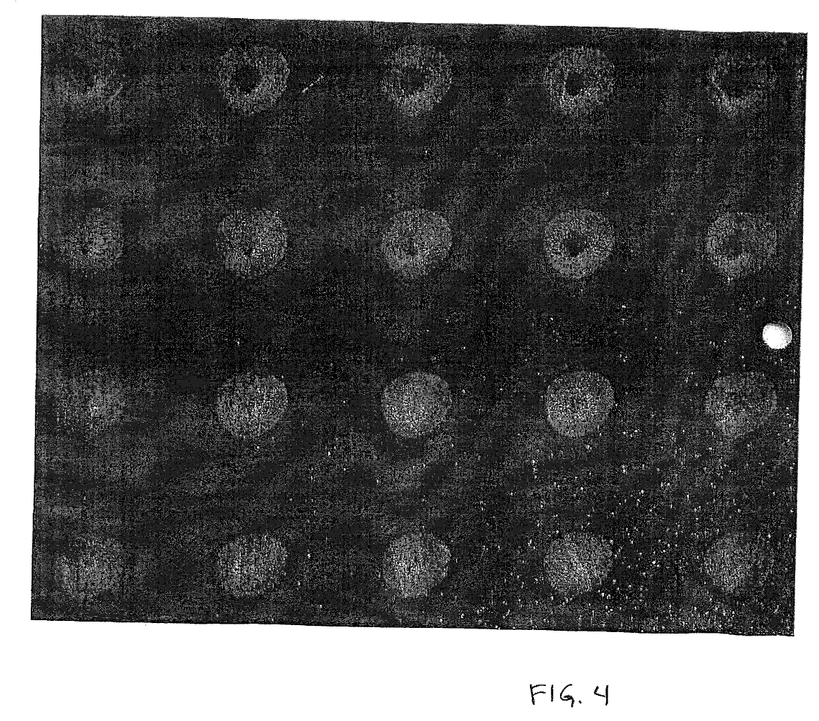

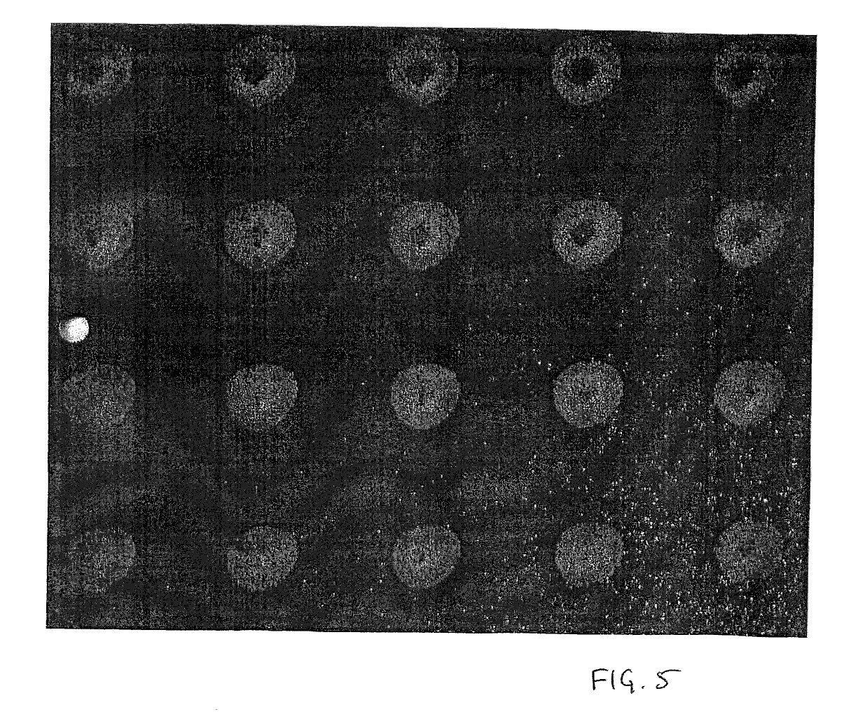

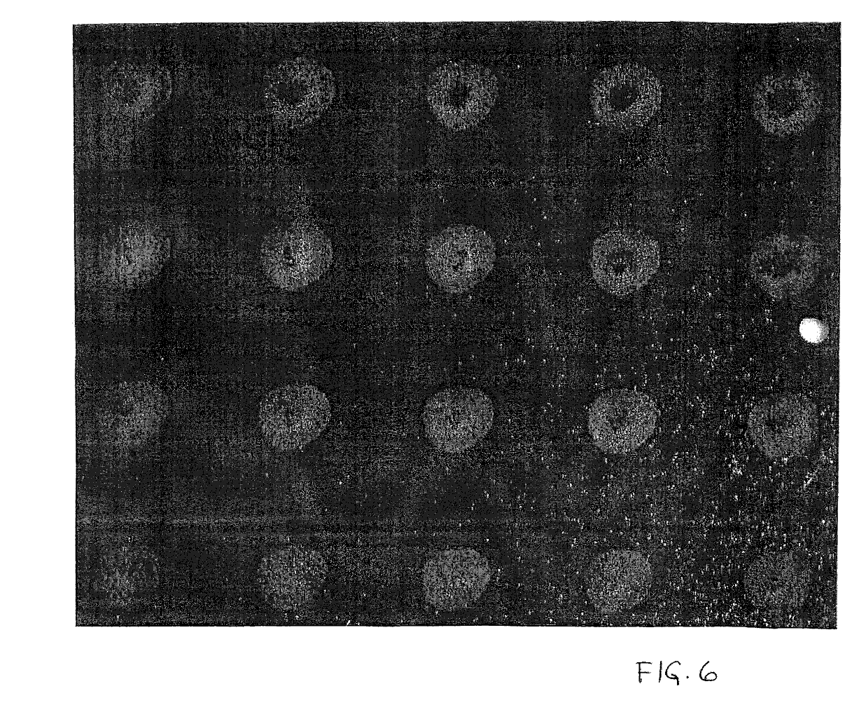

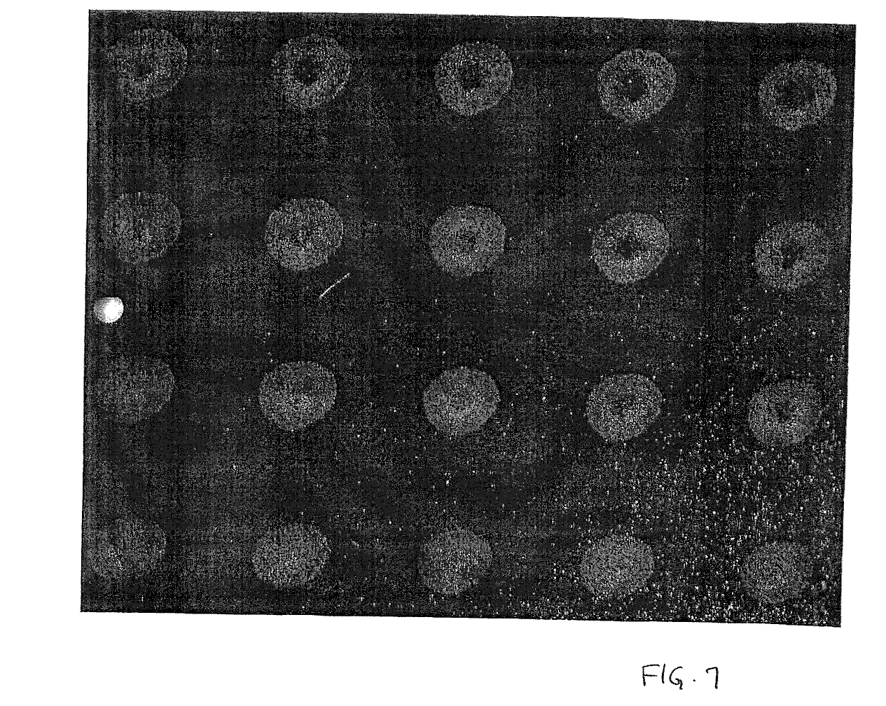

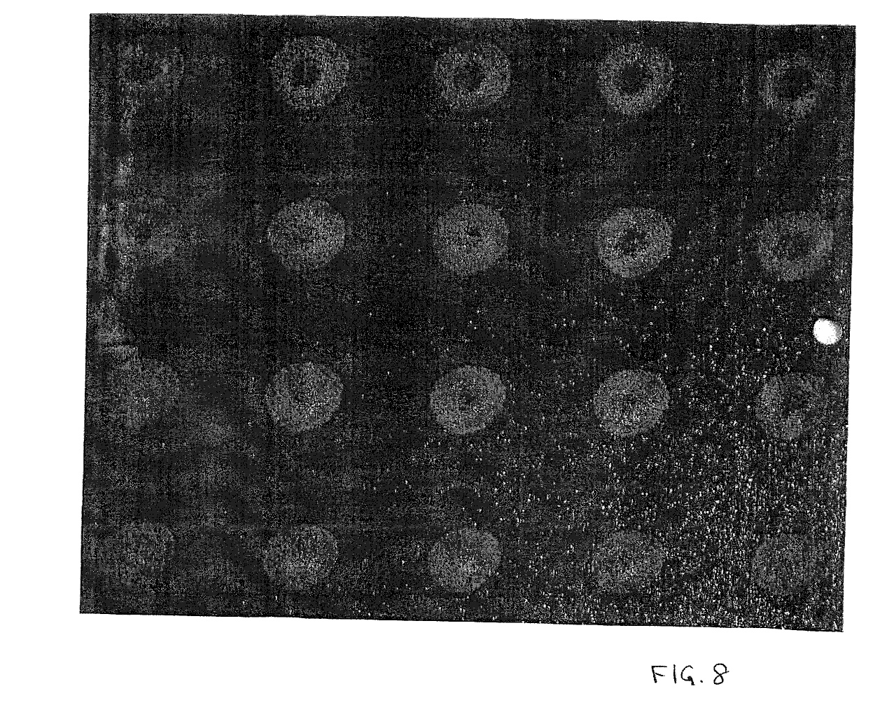

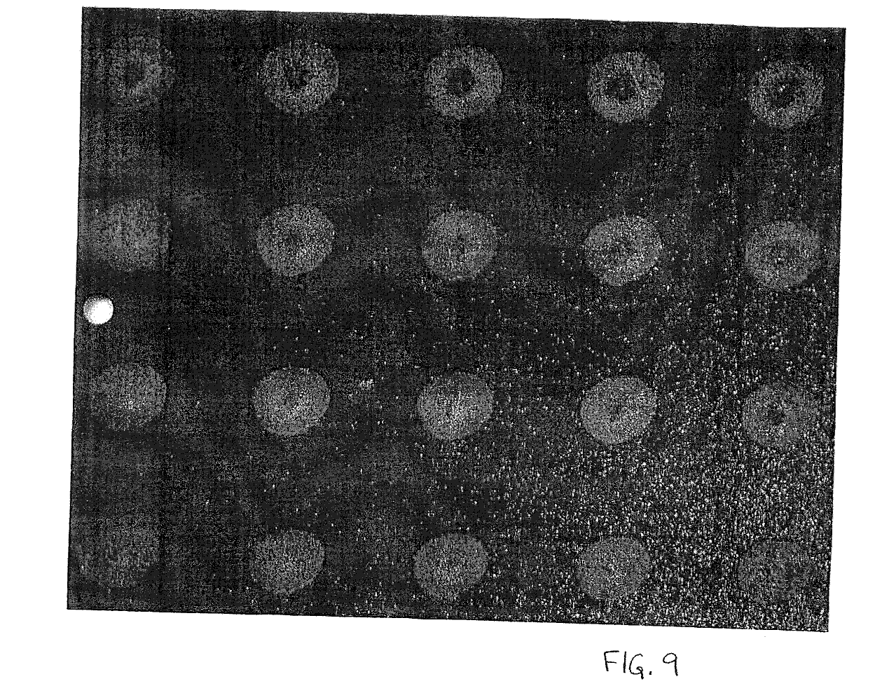

A breakthrough point is detected at which a smallest number of pulses was sufficient to penetrate through the polymer layer 15 and the upper layer 17, thereby exposing the lower layer to a detection of the second characteristic. In the exemplary embodiment discussed above, the breakthrough point occurs when the Mylar® layer 15 and the aluminum layer 17 have both been penetrated, exposing the black plastic layer 18 beneath the aluminum layer 17. When the film 14 has not been exposed to pulses, it appears black (see FIGS. 4-9); when the Mylar® layer 15 has been penetrated, the film 14 appears light-colored; when the Mylar® layer 15 and the aluminum layer 17 have both been penetrated, a black central portion appears. Continued delivery of pulses to a broken-through point will widen the central portion. A peak fluence of the excimer laser can then be determined from the number of pulses received at the detected breakthrough point.

The photographs of FIGS. 4-9 show the results of tests using the system 10 of the present invention. FIGS. 4-6 were performed at 100 Hz, with each spot having been ablated by one additional spot from the pulse to its immediate left. Each row was exposed to fewer pulses than the row above it. The tests of FIGS. 7-9 were performed at 400 Hz. Additional pulses were required to reach breakthrough compared with the 100-Hz test, since the beam shape changes at different repetition rates.

Among the benefits of the present invention is its speed. A grid of 15×15 pulses, with approximately 40 pulses at each point would take approximately 23 seconds, and would provide very high resolution. Preferably, a constant repetition rate is used, so that the laser energy remains as stable as possible over the duration of the test, reducing the effect of varying energy on the effective volume per shot that each pulse has.

Also preferably, the revisit times for the pattern comprises at least 0.5 sec, so that plume and thermal effects are avoided. When a location is to be skipped, the laser beam is preferably steered with the high-speed scanners to a “dump” location to allow the laser to continue to fire steadily, irrespective of whether the pulse is being steered to the film. Such a steady pulse rate provides the most stable average energy. The laser energy is also preferably retained in closed-loop control to ensure that the laser energy does not drift appreciably during the procedure.

In one embodiment, the film can be read by a human user; alternatively, the test can be automated with the use of image processing techniques to detect the breakthrough point and to “score” the patterns for areas of excess wear. Further, the size of the breakthrough hole can be measured, providing additional data for measuring the relative optics transmission of each point. The first two or three pulses following breakthrough will cause a measurable enlargement of each perforation hole.

Another aspect of the invention is directed to a system and method for characterizing an optical system for uniformity. In this method the excimer laser beam pulses are scanned in the predetermined pattern on a film when the Mylar® layer 15 and the aluminum layer 17 have both been penetrated, 14 as described above. The predetermined pattern can comprise a plurality of discrete points receiving a same number of pulses. The pulse number comprises a previously determined number known to be sufficient to penetrate through the polymer layer 15 and the upper layer 17, thereby exposing the lower layer 18 to a detection of the second characteristic (i.e., the black hole in the center of the ablated spot).

The film 14 can be examined to determine any points not having experienced a penetration through the polymer layer 15 and the upper layer 17. From this examination, a correlation can be made of the non-penetrated points with an area on the optical train to identify a region of non-uniformity thereon.

What is claimed is:

1. A method of determining a peak fluence of an excimer laser comprising the steps of:
scanning an excimer laser beam comprising a series of pulses in a predetermined pattern on a film comprising a polymer layer atop a substrate comprising an upper layer having a first characteristic and a lower layer beneath the upper layer having a second characteristic detectably distinct from the first characteristic, the predetermined pattern comprising a plurality of discrete points receiving incrementally greater numbers of pulses, at least one point receiving a sufficient number of pulses to penetrate through the polymer layer and the upper layer;
detecting a breakthrough point at which a smallest number of pulses was sufficient to penetrate through the polymer layer and the upper layer, thereby exposing the lower layer to a detection of the second characteristic; and
determining from the number of pulses received at the detected breakthrough point a peak fluence of the excimer laser.

2. The method recited in claim 1, wherein the upper layer of the film comprises a metal film and the lower layer comprises a stiff backing layer.

3. The method recited in claim 2, wherein the upper layer of the film comprises an aluminum film, the first characteristic comprises a reflective surface of the aluminum film, the lower layer comprises a plastic material, and the second characteristic comprises the plastic material having a color contrastive with the aluminum film surface.

4. The method recited in claim 3, wherein the detecting step comprises detecting a point having a dark center, indicating that the polymer layer and the upper layer have been broken through.

5. The method recited in claim 1, wherein the predetermined pattern comprises a row of points, each successive point receiving one more pulse than a preceding point.

6. The method recited in claim 5, further comprising the step of reconstructing a pulse shape by detecting a change in a shape of the exposed lower layer with an increasing number of pulses.

7. A method of characterizing an optical system for uniformity comprising the steps of:
directing an excimer laser beam comprising a series of pulses through an optical train adapted to scan a substrate in a predetermined pattern;
scanning the excimer laser beam pulses in the predetermined pattern on a film comprising a polymer layer atop a substrate comprising an upper layer having a first characteristic and a lower layer beneath the upper layer having a second characteristic detectably distinct from the first characteristic, the predetermined pattern comprising a plurality of discrete points receiving a same number of pulses, the pulse number comprising a previously determined number known to be sufficient to penetrate through the polymer layer and the upper layer, thereby exposing the lower layer to a detection of the second characteristic;
examining the film to determine any points not having experienced a penetration through the polymer layer and the upper layer; and
correlating the non-penetrated points with an area on the optical train to identify a region of non-uniformity thereon.

8. The method recited in claim 7, wherein the upper layer comprises a metal film and the lower layer comprises a stiff backing layer.

9. The method recited in claim 8, wherein the upper layer comprises an aluminum film and the lower layer comprises a dark-colored plastic material.

10. The method recited in claim 9, wherein the examining step comprises detecting a point having a dark center, indicating that the polymer layer and the upper layer have been broken through.

11. A system for determining a peak fluence of an excimer laser comprising:
a film comprising a polymer layer atop a substrate comprising an upper layer having a first characteristic and a lower layer beneath the upper layer having a second characteristic detectably distinct from the first characteristic;
an optical system for scanning an excimer laser beam comprising a series of pulses in a predetermined pattern on the film, the predetermined pattern comprising a plurality of discrete points receiving incrementally greater numbers of pulses, at least one point receiving a sufficient number of pulses to penetrate through the polymer layer and the upper layer;
means for detecting a breakthrough point at which a smallest number of pulses was sufficient to penetrate through the polymer layer and the upper layer, thereby exposing the lower layer to a detection of the second characteristic; and
means for determining from the number of pulses received at the detected breakthrough point a peak fluence of the excimer laser.

12. The system recited in claim 11, wherein the upper layer of the film comprises a metal film and the lower layer comprises a stiff backing layer.

13. The system recited in claim 12, wherein the upper layer of the film comprises an aluminum film, the first characteristic comprises a reflective surface of the aluminum film, the lower layer comprises a plastic material, and the second characteristic comprises the plastic material having a color contrastive with the aluminum film surface.

14. The system recited in claim 3, wherein the detecting means comprises means for detecting a point having a dark center, indicating that the polymer layer and the upper layer have been broken through.

15. The system recited in claim 11, wherein the predetermined pattern comprises a row of points, and the optical system is adapted to deliver to each successive point one more pulse than a preceding point.

16. The system recited in claim 15, further comprising means for reconstructing a pulse shape by detecting a change in a shape of the exposed lower layer with an increasing number of pulses.

17. A system for characterizing an optical system for uniformity comprising:
a film comprising a polymer layer atop a substrate comprising an upper layer having a first characteristic and a lower layer beneath the upper layer having a second characteristic detectably distinct from the first characteristic;
an optical train through which an excimer laser beam comprising a series of pulses can be directed for scanning a substrate in a predetermined pattern;
a processor having software resident thereon for directing the optical train to achieve the predetermined pattern, the predetermined pattern comprising a plurality of discrete points being delivered a same number of pulses, the pulse number comprising a previously determined number known to be sufficient to penetrate through the polymer layer and the upper layer, thereby exposing the lower layer to a detection of the second characteristic;
means for examining the film to determine any points not having experienced a penetration through the polymer layer and the upper layer; and
means for correlating the non-penetrated points with an area on the optical train to identify a region of non-uniformity thereon.

18. The system recited in claim 17, wherein the upper layer of the film comprises a metal film and the lower layer comprises a stiff backing layer.

19. The system recited in claim 8, wherein the upper layer of the film comprises an aluminum film and the lower layer comprises a dark-colored plastic material.

20. The system recited in claim 19, wherein the examining means comprises means for detecting a point having a dark center, indicating that the polymer layer and the upper layer have been broken through.
